(19) United States
(12) Patent Application Publication
(10) Pub. No.: US 2011/0045058 A1
(43) Pub. Date: Feb. 24, 2011

(54) TRANSFECTION REAGENT

(75) Inventor: Anny Usheva-Simidjiyska, Brookline, MA (US)

Correspondence Address: CLARK & ELBING LLP, 101 FEDERAL STREET, BOSTON, MA, 02110, US

(73) Assignee: Beth Israel Deaconess Medical Center, Boston, MA (US)

(21) Appl. No.: 12/743,692

(22) PCT Filed: Nov. 20, 2008
(86) PCT No.: PCT/US08/12932

§ 371 Date: Oct. 26, 2010

Related U.S. Application Data
(60) Provisional application No. 61/003,732, filed on Nov. 20, 2007.

(51) Int. Cl. A61K 9/127; C12N 5/071; C12N 5/07; C12N 5/04; C12N 5/10; A61K 31/7105; A61K 31/7088; A61K 38/16; A61K 38/00; A61K 31/70; A61P 43/00; A61K 38/43; A61K 35/76

(52) U.S. Cl. 424/450; 435/325; 435/348; 435/410; 435/419; 514/44 A; 514/44 R; 514/1.1; 514/21.2; 514/23; 424/94.1; 424/93.6

(57) ABSTRACT

The invention described herein features methods, compositions, and kits for introducing a nucleic acid molecule or biologically active molecule into a eukaryotic cell in vitro or in vivo utilizing a reagent containing energy-rich additives.

### BACKGROUND OF THE INVENTION

Efficient reagents for permeablizing cells to nucleic acid molecules for gene expression, gene transfer, and gene silencing are of great importance to medicine and biomedical science. Transfection, the transfer of molecules through the cell membrane, requires temporary disruption of the lipid bilayer and can be accomplished in different ways. Electroporation uses short pulses of electricity to disrupt the negatively charged lipid bilayer to form a pore facing the positive electrode. Heat-shock transfection utilizes “competent” cells pre-treated with calcium ions to render the membrane fragile to rapid changes in temperature. Chemical transfection uses lipid carrier compounds that incorporate the molecules to be transfected into micelles that fuse with the membrane.

An ideal transfection reagent would perform with uniformly high efficiency across a variety of cell types. However, differences in membrane composition and cell robustness to membrane disruption preclude the use of uniform transfection conditions for all cell types. In addition, higher efficiency of a reagent is usually counteracted by higher toxicity of the reagent, as larger and longer-lasting membrane pores increase the likelihood of damage to the cell. Thus, there exists a need in the art for a method and composition for improving the efficiency of transfection.

### SUMMARY OF THE INVENTION

The invention described herein features methods, compositions, and kits for introducing a nucleic acid molecule or biologically active molecule into a eukaryotic cell in vitro or in vivo utilizing a reagent containing energy-rich additives. The methods of the present invention include contacting the cell with a reagent that includes a nucleotide triphosphate (NTP), preferably adenosine triphosphate (ATP), contacting the molecule with the reagent, and incubating the cell with the molecule under conditions in which the molecule enters the cell. ATP may be present in the reagent of the methods, compositions, and kits of the invention described herein at a concentration of between, e.g., 0.5-20 mM, more preferably between, e.g., 1-9 mM. The reagent of the invention described herein may also include carbohydrates, salts, or buffering agents. The carbohydrate may include glucose at a concentration of between, e.g., 0.5-20 mM, more preferably between, e.g., 1-12 mM. The salt of the reagent may be, e.g., magnesium chloride, potassium phosphate, or sodium bicarbonate. Magnesium chloride may be at a concentration of between, e.g., 5-20 mM, more preferably between, e.g., 8-14 mM. Potassium phosphate may be at a concentration of between, e.g., 5-500 mM, more preferably between, e.g., 100-200 mM. Sodium bicarbonate may be present at a concentration of between, e.g., 1-50 mM, more preferably between, e.g., 12-31 mM. The buffering agent of the reagent may be, e.g., HEPES. The eukaryotic cell of the invention described herein may be, e.g., a mammalian cell, a human smooth muscle cell, a preadipocyte or adipocyte, a dividing cell or non-dividing cell, a transformed cell or primary cell, a somatic cell or stem cell, a plant cell, or an insect cell. The nucleic acid molecule of the methods, compositions, and kits of the present invention may be, e.g., DNA, RNA, DNA/RNA hybrids, or chemically modified nucleic acid molecules. The DNA may be, e.g., circular, linear, or a single-stranded oligonucleotide. The RNA may be, e.g., single-stranded (e.g., a ribozyme) or double-stranded (e.g., siRNA). The biologically active molecule may include, e.g., a nucleic acid, protein, peptide, carbohydrate, or organic compound. The biologically active molecule may also be, e.g., a therapeutic agent, diagnostic material, or research reagent.

The invention also features a kit, which includes a reagent containing a nucleotide triphosphate and instructions for introducing a nucleic acid molecule or biologically active molecule into a eukaryotic cell in vitro or in vivo using the reagent described herein.

In another embodiment, the invention features a method for improving the efficiency for introducing a nucleic acid molecule or biologically active molecule into a eukaryotic cell in vitro or in vivo, wherein the method includes contacting the cell with a reagent of the present invention, contacting the molecule with the reagent, and incubating the cell with the molecule under conditions in which the molecule enters the cell.

By “biologically active molecule” is meant any substance that can affect any physical or biochemical property of a biological organism, including, e.g., fungi, plants, animals, or humans. Examples of biologically active molecules include, e.g., peptides, proteins, enzymes, small molecule drugs, organic compounds, dyes, lipids, nucleosides, oligonucleotides, nucleic acids, viruses, liposomes, microparticles, and micelles.

By “buffering agent” is meant an agent, which, e.g., provides stable conditions for the storage of the reagent of the invention described herein. Any buffering agent not subjecting the nucleic acid or biologically active molecule to a condition of degradation may be used in the methods, compositions, and kits of the present invention. Representative buffering agents that may be used in the present invention include, e.g., N-[carbamoylmethyl]-2-aminoethanesulfonic acid (ACES), N-2[2-acetamido]-2-iminodiacetic acid (ADA), 2-amino-2-methyl-2,3-propanediol, 2-amino-2-methyl-1-propanol, 3-amino-1-propanesulfonic acid, 2-amino-2-methyl-1propanol, 3-[(1,1-dimethyl-2-hydroxyethypamino]-2-hydroxypropanesulfonic acid (AMSO), N,N-bis[2-hydroxyethyl]-2-aminoethanesulfonic acid (BES), N,N-bis[2-hydroxyethyl]glycine (BICINE), bis[2-hydroxyethyl]iminotris-[hydroxymethyl]methane (BIS-TRIS); 1,3-bis[tris(hydroxymethyl)-methylamino]propane (BIS-TRIS PROPANE), 4-[cyclohexylamino]-1-butanesulfonic acid (CABS), 3-[cyclohexylamino]-1-propanesulfonic acid (CAPS), 3-[cyclohexylamino]-2-hydroxy-1-propanesulfonic acid (CAPSO), 2-[N-cyclohexylamino]ethanesulfonic acid (CHES), 3-[N,N-bis(2-hydroxyethyl)amino]-2-hydroxypropanesulfonic acid (DIPSO), N-[2-hydroxy-ethyl]-piperazine-N′-[3-propanesulfonic acid] (HEPPS), N-[2-hydroxyethyl]piperazine-N′-[4-butanesulfonic acid] (HEPBS), N-[2-hydroxyethyl]piperazine-N′-[2-ethanesulfonic acid (HEPES), N-[2-hydroxyethyl]piperazine-N′-[2-hydroxypropanesulfonic acid] (HEPPSO), imidazole, 2-[N-morpholino]ethanesulfonic acid (MES), 4-[N-morpholino]butanesulfonic acid (MOBS), 3[N-morpholino]propanesulfonic acid (MOPS), 3-[N-morpholino]-2-hydroxypropanesulfonic acid (MOPSO), piperazine-N,N′-bis[2-ethanesulfonic acid] (PIPES), piperazine-N,N′-bis[2-hydroxypropanesulfonic acid (POPSO), N-tris[hydroxy-methyl]methyl-4-aminobutanesulfonic acid (TABS), N-tris[hydroxymethyl]methyl-3-aminopropanesulfonic acid (TAPS), 3-[N-tris(hydroxymethyl)methylamino]-2-hydroxy-propanesulfonic acid (TAPSO), triethanolamine (TEA), N-tris[hydroxymethyl]methyl-2-aminoethanesulfonic acid (TES), N-tris[hydroxymethyl]methylglycine (TRICINE), triethanolamine, tris[hydroxymethyl]aminomethane (TRIZMA) phosphate, acetate, citrate, borate, and bicarbonate.

By “carbohydrate” is meant a monosaccharide, disaccharide, oligosaccharide, or polysaccharide. Carbohydrates used in the methods, compositions, and kits of the present invention include, e.g., glucose, galactose, sucrose, trehalose, mannitol, fructose, maltose, raffinose, lactose, or glycogen, or any isomer or stereoisomer thereof.

By “eukaryotic cell” is meant a cell of any type and from any source having a nucleus. Types of eukaryotic cells include, e.g., epithelial, fibroblastic, neuronal, hematopoietic, muscle (e.g., human smooth muscle cells), adipocytic, or preadipocytic from primary cells, tumor cells, or transformed cell lines. Appropriate cell lines include, for example, COS, HEK293T, CHO, HepG2, HeLa, and NIH cell lines such as, e.g., NIH-3T3. The eukaryotic cell of the invention may be, e.g., a mammalian cell. The eukaryotic cell of the invention may also be, e.g., a dividing or non-dividing cell, or a somatic or stem cell. Sources of such cells include, e.g., a human, canine, mouse, hamster, cat, bovine, porcine, monkey, ape, sheep, fish, insect, fungus, or any plant (e.g., crop plant, ornamental, or tree).

By “introducing into a cell” is meant facilitating the uptake or absorption into the cell of a nucleic acid molecule or biologically active molecule through, e.g., transfection, as is understood by those skilled in the art. Absorption or uptake of the nucleic acid or biologically active molecule can occur through unaided diffusive or active cellular processes, or by auxiliary agents or devices. The meaning of this term is not limited to cells in vitro. A molecule may also be “introduced into a cell,” wherein the cell is part of a living organism. In such an instance, introduction into the cell will include the delivery to the organism. For example, in vivo delivery of the nucleic acid molecule can include, e.g., injection of the nucleic acid into a tissue site or systemic administration. In vitro introduction into a cell includes methods known in the art, such as, e.g., electroporation or chemical transfection (e.g., lipofection or heat-shock transfection).

By “kit” is meant a kit for transfection, which includes the reagent of the present invention. Such kits may comprise a carrying means being compartmentalized to receive in close confinement one or more container means such as, e.g., vials or test tubes. Each of such container means comprises components or a mixture of components needed to perform a transfection. Such kits may include, e.g., one or more components selected from nucleic acid molecules, cells, the reagent of the present invention, lipid-aggregate forming compounds, transfection enhancers, or biologically active molecules.

By “nucleic acid molecule” is meant a polymer of nucleotides, or a polynucleotide. The term is used to designate a single molecule, or a collection of molecules. A nucleic acid molecule may be, e.g., DNA (e.g., circular DNA, linear DNA, cDNA, genomic DNA, or plasmid DNA), RNA (e.g., siRNA, ribozymes, mRNA, rRNA, or tRNA), a DNA/RNA hybrid, a peptide nucleic acid, a nucleic acid vector, or a chemically modified nucleic acid molecule. The nucleic acid molecule may be single-stranded or double-stranded, and may include coding regions, non-coding regions, and regions of various control elements (e.g., promoters). The nucleic acid molecules may contain, e.g., natural or non-natural nucleobases. The concentration of the nucleic acid molecule of the present invention may be, e.g., between 0.1 pg/ml to 10 mg/ml.

By “nucleotide triphosphate” (NTP) is meant adenosine triphosphate (ATP), guanosine triphosphate (GTP), cytidine triphosphate (CTP), thymidine triphosphate (TTP), or uridine triphosphate (UTP), or an analog thereof.

By “salt” is meant an ionic compound used to provide an adequate concentration of essential inorganic ions necessary for the reagent of the present invention. The salts of the invention may include, e.g., calcium chloride, potassium phosphate, sodium bicarbonate, ferric nitrate, potassium chloride, magnesium sulfate, sodium chloride, sodium phosphate, Tris-HCl, Tris-EDTA, sodium acetate, potassium acetate, or magnesium acetate, or any other biologically compatible salt.

By “transfection” is meant the introduction of a nucleic acid molecule or biologically active molecule from directly outside a cell membrane to within the cell membrane, such that the molecule is expressed or has a biological function within the cell. Transfection may be facilitated through, e.g., electroporation or chemical transfection (e.g., using lipids or calcium phosphate). By “transfection efficiency” is meant the percentage of cells that have a given nucleic acid or biologically active molecule present within the cell after a certain period of time post-transfection.

### BRIEF DESCRIPTION OF THE FIGURES

FIG. 1 shows cell viability (diamonds) and siRNA transfection efficiency (bars) of the transfection reagent of the present invention compared to Amaxa™^{,} Nucleo*transfection buffer in human coronary artery smooth muscle cells (CASMC), HUVEC, and HeLa cells.

FIG. 2 shows the knock-down of transcription factor TFIIB expression in HeLa cells using the transfection reagent of the present invention. TFIIB protein was visualized with anti-TFIIB antibody immunostaining and cellular DNA was visualized through Hoechst staining. Panel (a) shows that transfection with the reagent results in greater than 95% of the cells expressing less TFIIB compared to untransfected cells in panel (b). Panel (b) is a control, showing the expression of endogenous TFIIB in the absence of TFIIB-specific siRNA. Panel (c) shows transfection with an Amaxa™ reagent, demonstrating that more than 20% of the cells have not been efficiently transfected and continue to express TFIIB.

### DETAILED DESCRIPTION OF THE INVENTION

The invention described herein features methods, compositions, and kits for introducing a nucleic acid molecule or biologically active molecule into a eukaryotic cell in vitro or in vivo utilizing a reagent containing energy-rich additives.

### Composition of the Reagent

The reagent of the invention described herein includes a nucleotide triphosphate (NTP). Preferably, the NTP is adenosine triphosphate (ATP). ATP may be present in the reagent at a concentration of, e.g., about 0.1, 0.5, 1, 2, 3, 4, 5, 10, 15, 20, or 50 mM. In a more preferred embodiment, ATP is present in the reagent at a concentration of, e.g., about 1, 2, 3, 4, 5, 6, 7, 8, or 9 mM. The reagent may further include, e.g., carbohydrates, salts, or buffering agents. Carbohydrates may include, e.g., glucose, galactose, sucrose, trehalose, mannitol, or lactose. Preferably, the carbohydrate is glucose. Glucose may be present in the reagent at a concentration of, e.g., about 0.1, 0.5, 1, 2, 3, 4, 5, 10, 15, 20, or 50 mM. In a preferred embodiment, glucose is present in the reagent at a concentration of about 1, 2, 3, 4, 5, 6, 7, 8, 9, 10, 11, or 12 mM. Salts present in the reagent may include, e.g., magnesium chloride, potassium phosphate, or sodium bicarbonate. Magnesium chloride may be present in the reagent at a concentration of, e.g., about 0.1, 0.5, 1, 2, 3, 4, 5, 10, 15, 20, or 50 mM. In a preferred embodiment, magnesium chloride is present in the reagent at a concentration of, e.g., about 8, 9, 10, 11, 12, 13, or 14 mM. Potassium phosphate may be present in the reagent at a concentration of, e.g., about 1, 5, 10, 25, 50, 75, 100, 150, 200, 250, 300, 350, 400, 450, or 500 mM. In a preferred embodiment, potassium phosphate is present in the reagent at a concentration of, e.g., about 100, 110, 120, 130, 140, 150, 160, 170, 180, 190, or 200 mM. Sodium bicarbonate may be present in the reagent at a concentration of, e.g., about 0.1, 0.5, 1, 2, 3, 4, 5, 10, 15, 20, or 50 mM. In a preferred embodiment, sodium bicarbonate is present at a concentration of 12, 13, 14, 15, 16, 17, 18, 19, 20, 21, 22, 23, 24, 25, 26, 27, 28, 29, 30, or 31 mM. The reagent may also contain a buffering agent (e.g., HEPES) at a concentration of, e.g., about 1, 5, 10, 20, 50, 100, 200, 250, or 500 mM at a pH of, e.g., about 6.5, 7, or 7.5.

The pH of the reagent may be adjusted using an acid (e.g., HCl) or a base (e.g., NaOH). The reagent may be filtered and/or sterilized. The reagent of the invention may be stored at a temperature of, e.g., about −80, −20, 0, 1, 2, 3, 4, 5, 6, 7, 8, 9, 10, 15, 20, 25, or 30° C. for, e.g., about 1 day, 2 days, 3 days, 5 days, 1 week, 2 weeks, 3 weeks, 1 month, 2 months, 4 months, 6 months, 1 year, or longer.

Additional components may be added to the reagent to facilitate the intracellular transport of the nucleic acid or biologically active molecule such as, e.g., polyethylenimine (PEI), polyalkylenimines, polyarginines, polyamines, protamines, polylysines, fusogenic peptides, polyamidoamine dendrimers, pegalated cationic polymers, cationic polymer conjugates (e.g., PEI-cholesterol or polylysine cholesterol), chitosans, cationic dextrans, cationic cyclodextrins, or cationic lipids (e.g., dioleoyltrimethyl-ammonium propane (DOTAP), N-[1(-2,3-dioleoyloxy)propyl]-N,N,N-trimethylammonium chloride (DOTMA), N,N-dioleyl-N,N-dimethylammonium chloride (DODAC), Lipofectamine™, or Gene Porter™).

The reagent described herein may provide a transfection efficiency of greater than about, e.g., 50, 55, 60, 65, 70, 75, 80, 85, 90, or 95%.

### Nucleic Acids and Other Biologically Active Molecules

A nucleic acid molecule used in the methods, compositions, and kits of the present invention may be, e.g., DNA (e.g., circular DNA, linear DNA, cDNA, genomic DNA, or plasmid DNA), RNA (e.g., siRNA, ribozymes, mRNA, rRNA, or tRNA), a DNA/RNA hybrid, a peptide nucleic acid, an oligonucleotide, a nucleic acid vector, or a chemically modified nucleic acid molecule. The nucleic acid molecule may be single-stranded or double-stranded, and may include coding regions, non-coding regions, and regions of various control elements (e.g., promoters).

The amount of nucleic acid utilized according to the methods, compositions, and kits of the present invention will vary greatly according to a number of factors including, e.g., the susceptibility of the target cells to nucleic acid uptake, the level of protein expression desired, if any, and the purpose of the transfection. For example, the amount of nucleic acid suitable for gene therapy in a human may be extrapolated from the amount of nucleic acid effective for gene therapy in an animal model. Furthermore, the amount of nucleic acid necessary for cell transfection will decrease with a corresponding increase in the efficiency of the transfection method used. In a preferred embodiment, the total concentration of the nucleic acid is from, e.g., about 0.1 pg/ml to about 15 mg/ml.

The nucleic acid molecule of the present invention may, e.g., be obtained from natural sources, be produced recombinantly, or be made through chemical synthesis (e.g., polymerase chain reaction (PCR)). Suitable plasmid DNA molecules may be isolated from bacteria using conventional plasmid purification techniques, which minimize contaminating RNA molecules and endotoxins. Genomic DNA may be isolated from cells using conventional DNA isolation techniques, which typically require sodium hydroxide or enzymatic lysis followed by phenol-chloroform extraction, ethanol precipitation, or affinity chromatography to isolate the DNA. Customized synthetic nucleic acids that are suitable in the present invention are also available commercially from suppliers.

The nucleic acid molecules used may include those encoding and capable of expressing therapeutic or otherwise useful proteins in cells, those which inhibit undesired expression of nucleic acids in cells, those which inhibit undesired enzymatic activity or activate desired enzymes, those which catalyze reactions (e.g., ribozymes), or those which function in diagnostic assays. The nucleic acid molecule may be modified, e.g., to possess a specific function (e.g., a nuclear targeting nucleic acid molecule). The nucleic acid may, e.g., be suitable for use in gene therapy, gene vaccination, or in anti-sense therapy, or the nucleic acid molecule may be or may relate to a gene that is the target for a particular gene therapy, gene vaccination, or anti-sense therapy.

The results of nucleic acid delivery into the eukaryotic cell of the invention may be analyzed by different methods known to one skilled in the art (see, e.g., U.S. Pat. Nos. 6,458,026, 7,056,741, and 7,125,709, hereby incorporated by reference). In the case of gene transfection and antisense nucleic acid delivery, the target gene expression level may be detected by reporter genes (e.g., green fluorescent protein (GFP) gene expression, luciferase gene expression, or (β-galactosidase gene expression). The signal of GFP can, e.g., be directly observed under a fluorescence microscope, the activity of luciferase can, e.g., be detected by a luminometer, and the blue product catalyzed by β-galactosidase can, e.g., be observed under a microscope or determined by a microplate reader. The target modulated by the nucleic acid molecule delivered to the cell according to methods described herein can be monitored by various methods, such as, e.g., detecting immunofluorescence or enzyme immunocytochemistry, autoradiography, or in situ hybridization. If immunofluorescence is used to detect the expression of a protein encoded by the nucleic acid, a fluorescently labeled antibody that binds to the target protein may be used. Cells containing the protein are then identified by detecting a fluorescent signal. If the delivered nucleic acid molecule, e.g., modulates gene expression, the target gene expression level can also be determined by methods such as, e.g., autoradiography, in situ hybridization, in situ PCR, or by any other method known to one of skill in the art.

The methods, compositions, and kits provided herein can also be readily adapted in view of the disclosure provided herein to introduce biologically active molecules or substances other than nucleic acid molecules into a eukaryotic cell, including, e.g., polyamines, polyamine acids, peptides, proteins, biotin, carbohydrates, and organic compounds. Other useful materials (e.g., therapeutic agents, diagnostic materials, or research reagents) may be introduced into the eukaryotic cell of the invention by the methods described herein. The amount of biologically active molecule utilized according to the methods, compositions, and kits of the present invention will vary greatly according to a number of factors including, e.g., the susceptibility of the target cells to the biologically active uptake. The total concentration of the biologically active molecule may be from, e.g., about 0.1 pg/ml to about 100 mg/ml.

### Eukaryotic Cell

Cultured eukaryotic cells used in the invention may be grown and maintained in, e.g., Dulbecco's Modified Eagles Medium (DMEM) containing 10% heat-inactivated fetal bovine serum (FBS) with L-glutamine and penicillin/streptomycin. It will be appreciated by those of skill in the art that certain cells should be cultured in a special medium, as some cells require special nutrition (e.g., growth factors and amino acids). The optimal density of cells depends on the cell type and the purpose of the experiment. For example, a population of 55, 60, 65, 70, 75, 80, or 85% confluent cells is preferred for gene transfection, but the optimal condition for oligonucleotide delivery is 20, 25, 30, 35, 40, or 45% confluent cells.

### Transfection

Methods of transfection are well known in the art (see, e.g., U.S. Patent Nos. 5,763,240, 6,806,084, 6,812,204, 6,989,434, and 7,056,741, and U.S. Patent Application Publication Nos. 2006/0229246 and 2007/0254358, hereby incorporated by reference).

The eukaryotic cell and/or nucleic acid or biologically active molecule of the invention may be contacted with the reagent described herein prior to, during, and after transfection. The cell and molecule are incubated together in the presence of the reagent under optimal transfection conditions for the cell type (e.g., 37° C. and 5-10% CO₂). The cells may be incubated, e.g., on a transfection plate, a 96-well plate, or any other suitable plate or dish for the given cell type. Alternatively, the molecule and reagent may be administered to a cell in vivo for, e.g., therapeutic purposes. The incubation time is dependent on, e.g., the purpose of experiment and the cell type. The cells may be incubated with the nucleic acid or biologically active molecule for 2, 4, 6, 12, 24, 48, or 72 hours, or for shorter or longer durations. Minutes to several hours of incubation may be required for certain experiments, and the cells may be observed at defined time points. The efficiency of the transfection using the reagent described herein may be greater than 50, 55, 60, 65, 70, 75, 80, 85, 90, or 95%.

Transfection through, e.g., electroporation may be used in both in vitro and in vivo procedures to introduce nucleic acid molecules into cells. With in vitro applications, a sample of cells is mixed with the molecule of interest in the presence of the transfection reagent described herein and placed between electrodes (e.g., parallel plates). The electrodes then apply an electrical field to the cells. Examples of systems that perform in vitro electroporation include the Electro Cell Manipulator ECM600 and the Electro Square Porator T820, both made by Genetronics, Inc. (see, e.g., U.S. Pat. Nos. 5,869,326 and 6,812,204, hereby incorporated by reference).

The transfection methods of the present invention employing the reagent described herein may be applied to in vitro and in vivo transfection of cells. The methods of this invention are useful as a step in any therapeutic method requiring the introduction of nucleic acids or other molecules into cells. In particular, these methods are useful in cancer treatment, in in vivo and ex vivo gene therapy, and in diagnostic methods. The transfection compositions of this invention can be employed as research reagents in any transfection of cells for research purposes.

### EXAMPLES

The following example is provided for the purpose of illustrating the invention and is not meant to limit the invention in any way.

### Example 1

### Preparation of the Transfection Reagent

The reagent of this example was prepared for introducing a nucleic acid molecule or biologically active molecule into a eukaryotic cell (Table 1).

TABLE 1

| Ingredient | Amount |
|---|---|
| Adenosine triphosphate (ATP) | 1-9 mM |
| Magnesium chloride (MgCl₂) | 8-14 mM |
| Glucose | 1-12 mM |
| Potassium phosphate (KH₂PO₄) | 100-200 mM |
| Sodium bicarbonate (NaHCO₃) | 12-31 mM |

The reagent was prepared by combining the ingredients of Table 1. The ATP, MgCl₂, glucose, KH₂PO₄, and NaHCO₃ may be purchased from Sigma-Aldrich (St. Louis, Mo., U.S.A.). A buffer (e.g., HEPES) may also be added to the reagent. The reagent may be dispensed into aliquots and stored at 4° C. for 2 months.

The reagent is used for the introduction of a molecule into a eukaryotic cell using transfection methods known to one of skill in the art. A sufficient volume of the reagent is supplied to the eukaryotic cell (e.g., in vitro or in vivo) and/or the molecule for the transfection. The efficiency of transfection of, e.g., an siRNA, is, e.g., greater than 95% in, e.g., human smooth muscle cells from saphenous veins and coronary, mammary, and iliac arteries.

### Example 2

### Comparison of Transfection Reagents

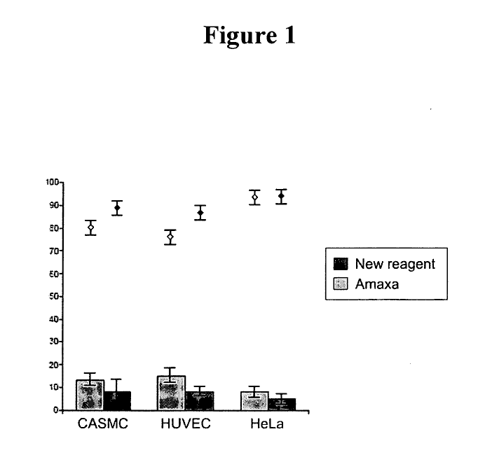

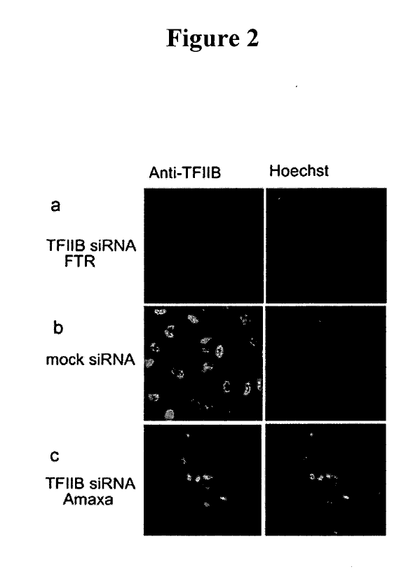

It is known that, following electroporation, the cell membrane remains porous for as long as several minutes post-electroporation, and it has been proposed that membrane healing is an active cellular process that requires energy. Accordingly, a transfection reagent was developed that minimizes cell mortality by stimulating membrane repair. A buffered solution containing molecules to support the energy-costly transport across the membrane and membrane repair was prepared (see, e.g., Example 1, above). It was demonstrated that cellular electroporation of plasmid DNA and siRNA was improved in transfection efficiency and cell survival when compared to Amaxa™ transfection buffers (FIGS. 1 and 2). The reagent may be utilized for, e.g., electroporation or as additive in chemical transfection protocols.

### Other Embodiments

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1-29. (canceled)

30. A reagent for introducing a nucleic acid molecule or biologically active molecule into a eukaryotic cell in vitro or in vivo, said reagent comprising a nucleotide triphosphate (NTP) and further comprising at least one of carbohydrates, salts, or buffering agents.

31. The reagent of claim 30, wherein said NTP is adenosine triphosphate (ATP).

32. The reagent of claim 31, wherein said ATP is at a concentration of between 0.5-20 mM.

33-34. (canceled)

35. The reagent of claim 30, wherein said carbohydrate is glucose.

36. The reagent of claim 35, wherein said glucose is at a concentration of between 0.5-20 mM.

37. (canceled)

38. The reagent of claim 30, wherein said salt is magnesium chloride, potassium phosphate, or sodium bicarbonate.

39. The reagent of claim 38, wherein said magnesium chloride is at a concentration of between 5-20 mM.

40. (canceled)

41. The reagent of claim 38, wherein said potassium phosphate is at a concentration of between 5-500 mM.

42. (canceled)

43. The reagent of claim 38, wherein said sodium bicarbonate is at a concentration of between 1-50 mM.

44. (canceled)

45. The reagent of claim 30, wherein said buffering agent is HEPES.

46. The reagent of claim 30, wherein said eukaryotic cell is a mammalian cell, a plant cell, or an insect cell.

47. The reagent of claim 46, wherein said mammalian cell is a human smooth muscle cell.

48. The reagent of claim 30, wherein said eukaryotic cell is a preadipocyte or adipocyte.

49. The reagent of claim 30, wherein said eukaryotic cell is a dividing cell or non-dividing cell.

50. The reagent of claim 30, wherein said eukaryotic cell is a transformed cell or primary cell.

51. The reagent of claim 30, wherein said eukaryotic cell is a somatic cell or stem cell.

52-53. (canceled)

54. The reagent of claim 30, wherein said nucleic acid molecule is selected from the group consisting of DNA, RNA, DNA/RNA hybrids, and chemically modified nucleic acid molecules.

55-57. (canceled)

58. The reagent of claim 54, wherein said RNA is siRNA.

59-122. (canceled)

123. The reagent of claim 30, wherein said biologically active molecule is a nucleic acid, protein, peptide, carbohydrate, or organic compound.

124. The reagent of claim 30, wherein said biologically active molecule is a therapeutic agent, diagnostic material, or research reagent.

125-130. (canceled)
